(19) United States
(12) Patent Application Publication
(10) Pub. No.: US 2023/0191395 A1
(43) Pub. Date: Jun. 22, 2023

(54) MEDICAL DIAGNOSTIC CHIP AND METHOD FOR MANUFACTURING THE MEDICAL DIAGNOSTIC CHIP

(71) Applicant: KYUNGPOOK NATIONAL UNIVERSITY INDUSTRY-ACADEMIC COOPERATION FOUNDATION, Daegu (KR)

(72) Inventor: Sung-Wook NAM, Daegu (KR)

(21) Appl. No.: 17/802,667

(22) PCT Filed: Oct. 14, 2020
(86) PCT No.: PCT/KR2020/013998

§ 371 Date: Sep. 8, 2022

(30) Foreign Application Priority Data
Oct. 14, 2019 (KR) 10-2019-0127217

(51) Int. Cl. B01L 3/00; G03F 7/20; B82Y 5/00

(52) U.S. Cl. CPC B01L 3/502707; B82Y 5/00; G03F 7/7045; G03F 7/70633

(57) ABSTRACT

Provided is a method for manufacturing a medical diagnostic chip using a mixed lithography method. The method includes forming first patterns in a first region using first lithography, and forming second patterns in a second region using second lithography, wherein a part of the second patterns is formed in a part of the first region among the region where the first region and the second region are adjacent to each other.

### TECHNICAL FIELD

The present invention relates to a medical diagnostic chip and a method for manufacturing a medical diagnostic chip, and more particularly, to a method for manufacturing a medical diagnostic chip using mixed lithography.

### BACKGROUND ART

Exosomes are endoplasmic reticulum substances (extracellular vesicles, hereinafter, “EVs”) secreted from cells and may be used as cancer-specific biomarkers. The exosomes are known to have sphere shapes with sizes of 50 nm to several hundred nanometers. It has been known that the exosomes have different secretory mechanisms of the exosomes and exosomal proteins and miRNAs contained in the exosomes depending on normal cells or cancer cells, and that components thereof vary according to a size thereof.

A method using a deterministic lateral displacement (DLD) structure is a method that may sort particles dissolved in a fluid by size using a micropillar or nanopillar structure. By using this, in U.S. Princeton University and IBM Research Institute, there is a case in which an exosome isolation device is manufactured using the nanopillar structure.

In the method of manufacturing the exosome isolation device chip in the above case, a silicon ship was covered with Borosilicate glass and then a wafer bonding was performed. When the chip is manufactured using such method, the state of the chip is non-uniform, and particularly, it is known that the leakage of the fluid is serious, and it was difficult to commercialize the device manufactured by such method.

### SUMMARY OF THE INVENTION

The present invention has been made in an effort to easily manufacture a medical diagnostic chip capable of isolating exosome biomarkers from the human blood and sorting the isolated exosome biomarkers by size.

The problem to be solved by the present invention is not limited to the above-mentioned problems. The problems not mentioned will be clearly understood by those skilled in the art from the present specification and the accompanying drawings.

An exemplary embodiment of the present invention provides a method for manufacturing a medical diagnostic chip using a mixed lithography method.

The method may include forming first patterns in a first region using first lithography, and forming second patterns in a second region using second lithography, wherein a part of the second patterns is formed in a part of the first region among the region where the first region and the second region are adjacent to each other.

The first lithography may be electron beam lithography, and the second lithography may be photolithography.

The forming first patterns in the first region using the electron beam lithography may comprise forming nanopillar pattern.

The forming of the first patterns in the first region using the electron beam lithography may be forming the patterns using a positive electron beam resist.

The forming of the second patterns in the partial region of the first region in the region where the first region and the second region are adjacent to each other may be forming the patterns so that the second patterns using the second lithography are overlapped with an edge portion of the first region in the region where the first region and the second region are adjacent to each other.

The forming of the second patterns in the second region using the second lithography may be forming the patterns using a positive or negative photoresist.

Another exemplary embodiment of the present invention provides a method for manufacturing a medical diagnostic chip.

The forming a part of second patterns in the portion of the first region among the region where the first region and the second region are adjacent to each other comprises forming the second patterns using the second lithography as to overlap with an edge portion of the first region among the region where the first region and the second region are adjacent to each other.

The forming of the first patterns in the first region using the electron beam lithography may be forming the patterns in the form of having nanoholes.

The forming of the first patterns in the first region using the electron beam lithography may be forming the first patterns using a positive electron beam resist.

The forming of the second patterns so that the formed first region is protected may be forming the patterns so that the second patterns formed by using the second lithography do not affect the first patterns formed in the first region.

The forming of the second patterns in the second region using the second lithography may be forming the patterns using a positive or negative photoresist.

The method for manufacturing the medical diagnostic chip may further include pouring a polymer material into the formed chip; waiting for a predetermined time until the polymer material is hardened; and inverting the hardened polymeric material.

According to the present invention, it is possible to easily manufacture a medical diagnostic chip capable of isolating exosome biomarkers from the human blood and sorting the isolated exosome biomarkers by size.

According to the present invention, it is possible to manufacture a nanofluidic device chip in which nanopillar structures are arranged using a sacrificial process capable of manufacturing a nanofluidic device in a semiconductor FAB without secondary operations such as wafer bonding.

The effect of the present invention is not limited to the foregoing effects. Non-mentioned effects will be clearly understood by those skilled in the art from the present specification and the accompanying drawings.

### BRIEF DESCRIPTION OF THE DRAWINGS

FIGS. 1A to 1D are examples for describing a device isolation method using a DLD structure.

FIGS. 2A to 2H are diagrams sequentially illustrating a method for manufacturing a medical diagnostic chip according to an exemplary embodiment of the present invention.

FIG. 3 is a diagram illustrating a medical diagnostic chip manufactured according to the exemplary embodiment in FIG. 2.

FIGS. 4A and 4B show images in which photoresist patterns are arranged on electron beam lithography patterns.

FIGS. 5A to 5C illustrate results of confirming mixed patterns by microscopy.

FIGS. 6A and 6B are results confirmed by magnifying a middle mixed portion by microscopy when a manufacturing process of the medical diagnostic chip according to the present invention is terminated.

FIGS. 7A to 7H are diagrams sequentially illustrating a method for manufacturing a medical diagnostic chip according to another exemplary embodiment of the present invention.

FIG. 8 is a diagram illustrating a medical diagnostic chip manufactured according to the exemplary embodiment in FIG. 7.

FIG. 9 is a diagram for describing a process of FIG. 7E in more detail.

FIGS. 10A to 10C illustrate results in which electron beam lithography patterns and photoresist patterns are arranged.

FIGS. 11A to 11C illustrate results of confirming mixed patterns by microscopy.

FIGS. 12A and 12B illustrate a result of confirming a nanohole structure by microscopy and a result of observing the depths of nanoholes.

### DETAILED DESCRIPTION

Hereinafter, exemplary embodiments of the present invention will be described more fully hereinafter with reference to the accompanying drawings, in which exemplary embodiments of the invention are shown. However, the present invention can be variously implemented and is not limited to the following exemplary embodiments. In the following description of the present invention, a detailed description of known functions and configurations incorporated herein is omitted to avoid making the subject matter of the present invention unclear. In addition, the same reference numerals are used throughout the drawings for parts having similar functions and actions.

It will be understood that the terms “comprises,” “comprising,” “includes,” “including,” “have,” “having,” etc., when used in this specification, specify the presence of stated features, integers, steps, operations, elements, and/or components, but do not preclude the presence or addition of one or more other features, integers, steps, operations, elements, components, and/or groups thereof.

. As used herein, the singular forms “a”, “an” and “the” are intended to include the plural forms as well, unless the context clearly indicates otherwise. Further, shapes, sizes, and the like of the elements in the drawing may be exaggerated for clearer description.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings.

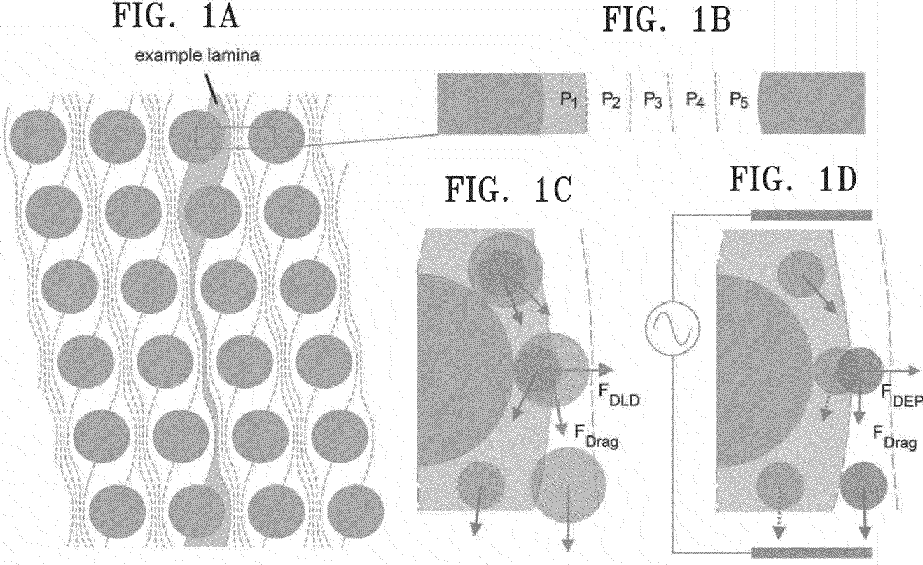

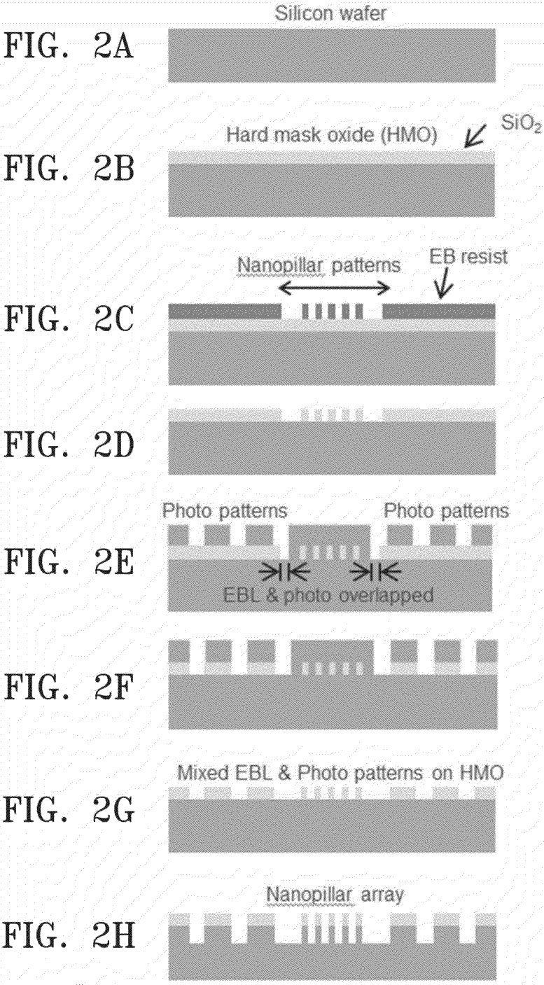

FIGS. 1A to 1D are examples for describing a device isolation method using a DLD structure.

FIG. 1A is a diagram for describing a principle of the DLD structure. FIG. 1B is a diagram schematically illustrating a concept of a laminar flow in the DLD structure. FIG. 1C is a diagram illustrating a principle of sorting large-sized particles and small-sized particles in the DLD structure. FIG. 1D is a diagram illustrating an effect when an AC voltage or current is applied from the outside in the DLD structure.

FIG. 1 is a diagram extracted from Lab on a Chip (2014) 14, 4139 and Lab on a Chip (2009) 9, 2698-2706.

Referring to FIG. 1A, a characteristic of the DLD structure is that an array of nanometer or micrometer-sized pillars is slightly inclined with respect to a vertical direction in which a fluid flows. The sizes of the pillars formed in the DLD structure are formed to be similar to the particle sizes of particles to be isolated through the chip of the present invention. For example, since EVs have sizes of 50 nm to several hundred nanometers, the sizes of pillars for isolating and sorting EVs may also have sizes of several tens to several hundred nanometers.

If the array of the pillars is not inclined, biomolecules flow uniformly according to the flow of the fluid regardless of sizes thereof, which is called a laminar flow. When the array of the pillars is inclined with respect to the direction (vertical direction) in which the fluid flows as shown in FIG. 1A, relatively large-sized molecules collide with the pillars and deviate from the laminar flow to be isolated in the direction in which the pillar array is inclined. This may be confirmed in FIG. 1B.

When the nanopillars are inclined as illustrated in FIG. 1, large-sized particles may flow in the direction in which the pillars are inclined, but small-sized particles may flow in the same direction as the fluid. That is, it is possible to sort particles by size through the inclined nanopillars, and it is possible to isolate and sort particles having various sizes by adjusting the sizes of the nanopillars. This may be confirmed in FIG. 1C.

FIG. 1D is a diagram illustrating an effect when an AC voltage or current is applied from the outside in the DLD structure. As shown in the example in FIG. 1D, there is an effect of controlling the flow of particles by applying the AC voltage to both ends of the nanopillar structure.

In order to manufacture the DLD structure as illustrated in FIG. 1, nanometer-sized pillars need to be arranged to be inclined with respect to the direction in which the fluid flows.

In the present invention, a medical diagnostic chip may be manufactured by a combination of a first lithography method and a second lithography method. In the present invention, patterns are formed on a substrate by mixing the first lithography method and the second lithography method, so that patterns having nanometer size and patterns having micrometer size may be formed on a single substrate. As a result, there is an effect that may be used as the medical diagnostic chip.

When the DLD structure is formed on the substrate by a conventional method, there is a problem in that the amount of a sample to be treated is too low and the efficiency is lowered. In the present invention, since a plurality of patterns may be formed on one substrate using mixed lithography, there is an effect that the treating efficiency may be increased.

More specifically, in the case of electron beam lithography, patterns having nanometer-level small sizes may be implemented, but the throughput is too low, so that it is very difficult to implement the patterns on a large area. In contrast, in the case of photolithography, it is easy to manufacture the patterns on the large area due to its high throughput, but it may be difficult to implement patterns having nanometer-level small sizes. In the present invention, since the patterns are formed on the substrate by mixing electron beam lithography and photolithography, there is an effect of simultaneously implementing patterns having nanometer-level small sizes and patterns having relatively large sizes on a large area.

There is a difference in protein components between EVs from cancer patients’ blood and EVs from normal humans, and when this difference is detected, it is possible to diagnose early whether or not to have cancer through a simple method.

In the description, it has been described that the medical diagnostic chip according to the present invention may be used for the method for early diagnosis of cancer cells. However, the use of the medical diagnostic chip manufactured according to the present invention is not limited to the exemplary embodiment, and may be variously used to sort various sizes of particles.

Hereinafter, a method of manufacturing the DLD structure on the substrate will be described in detail with reference to the drawings.

FIGS. 2A to 2H are diagrams sequentially illustrating a method for manufacturing a medical diagnostic chip according to an exemplary embodiment of the present invention.

Referring to FIG. 2A, there is disclosed a substrate used as a base of the medical diagnostic chip. The substrate may also be any material such as metal, glass, or resin. For example, as the material of the substrate, silicon, silicon carbide, gallium arsenide, spinel, indium phosphide, gallium phosphide, aluminum phosphide, gallium nitride, indium nitride, aluminum nitride, zinc oxide, magnesium oxide, aluminum oxide, titanium oxide, sapphire, quartz or pyrex may be used, but the present invention is not limited to these materials.

Referring to FIG. 2B, an oxide film may be formed on the substrate provided as the base. In the process of FIG. 2B, a uniform oxide film (SiO₂) may be formed by heating at a high temperature of 800 to 1200° C. or higher. The oxide film formed at this time may be used as a hard mask during an etching process in the first lithography and the second lithography to be described below.

Referring to FIG. 2C, first patterns may be formed on the oxide film by using the first lithography. The first lithography may be electron beam lithography. The first patterns may be electron beam patterns.

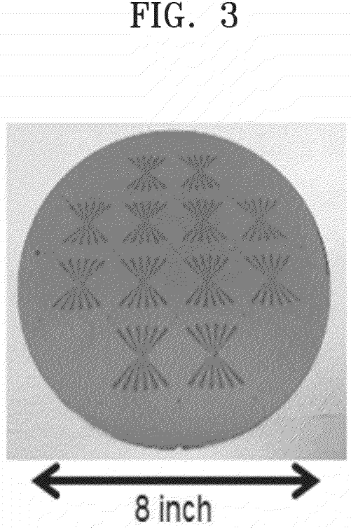

Referring to FIG. 2C, nanopillar patterns may be formed on the oxide film by using the first lithography. According to FIG. 2C, the nanopillar patterns may be formed on a first region by using the electron beam lithography. A plurality of first regions may be provided. Referring to FIG. 3 to be described below, the number of the first regions may be 12.

According to the characteristics of the electron beam lithography, a very small nanometer-sized pillar structure having sizes of several nanometers to several hundreds of nanometers may be manufactured in a limited region.

In the case of forming the nanopillar patterns using the electron beam lithography according to FIG. 2C, the electron beam patterns may be formed using an electron beam resist. In this case, the electron beam resist may be a positive resist.

According to FIG. 2D, etching may be performed on the electron beam patterns. When the etching is performed, a part of the silicon oxide film not covered by the electron beam patterns are etched away.

The etching includes a dry etching process or a wet etching process. In the present invention, the etching is mainly a dry etching process, and among them, reactive ion etching (RIE) may be used.

The etching in FIG. 2D may use reactive ion etching (RIE).

Although not illustrated in FIG. 2D, after etching process for the exposed oxide film, a strip process for removing the electron beam patterns may be performed.

When the processes of FIGS. 2A to 2D are completed, the first lithography process for the first region on the substrate is completed. That is, the electron beam patterns are formed in the first region. In order to form microchannels in addition to the nanopillar structure, a photoresist process may be additionally performed.

Referring to FIG. 2E, second patterns may be formed in a second region using a second lithography method. The second lithography method may be a photolithography method. The second patterns may be photo patterns. A nano-micro structure may be manufactured over a wide region of several mm or higher by using a photolithography method. In the case of photoresists, both positive and negative photoresists are possible.

The second region and the first region may be different regions. According to an example, the second region may be disposed to connect the plurality of first regions. A plurality of second regions may be provided. According to an example, the second regions may be disposed to be connected to both ends of the first region.

Referring to FIG. 2E, photo patterns are formed in the second region using the second lithography method.

A technical feature to be noted at this time is related to pattern formation in the second lithography in a region where the first region and the second region are adjacent to each other.

The photo patterns in the region where the first region and the second region are adjacent to each other need to be formed to be overlapped with the electron beam patterns formed in the previous step.

According to an example, at a point where the first region in which the electron beam patterns are formed and the second region in which the photo patterns are formed are adjacent to each other, the photo patterns may be partially overlapped with the edge portion of the first region. The photo patterns overlapped with the first region may be formed without overlapping with the nanopillar patterns formed on the first region.

The first region is a region where the nanopillar patterns illustrated in FIG. 2C are formed. The second region may be the remaining region other than the first region where the nanopillar patterns are formed.

That is, according to FIG. 2E, the photo patterns are formed in a portion overlapped with the edge portion of the nanopillar patterns on which the electron beam patterns are already formed to form the patterns so that the electron beam patterns and the photo patterns may be naturally connected to each other on one substrate.

Referring to FIG. 2F, a process of performing etching according to the formed photo patterns is disclosed.

Referring to FIG. 2G, a process of performing a stripping process of removing the photoresist is started after the etching is completed.

According to FIG. 2H, the depths of the nanopillar structure and the micropillar structure may be adjusted by further etching the substrate. FIG. 2H may be an optional step.

Hereinafter, the effect of the medical diagnostic chip manufactured according to the exemplary embodiment of FIG. 2 will be described through various drawings.

FIG. 3 is a diagram illustrating a medical diagnostic chip manufactured according to the exemplary embodiment in FIG. 2. Referring to FIG. 3, it can be seen that mixed lithography of a total of 12 photo patterns and electron beam patterns is formed on one substrate.

Like an exemplary embodiment of FIG. 3, mixed lithography of various numbers of photo patterns and electron beam patterns may be formed on one substrate.

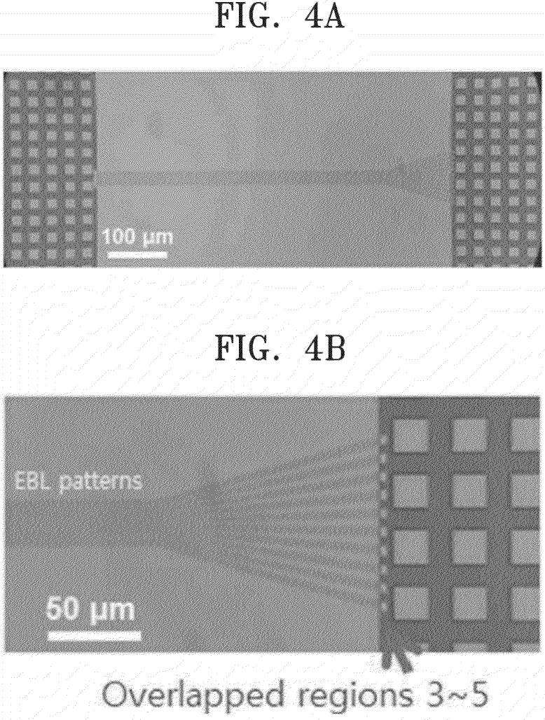

FIGS. 4A and 4B show images in which photoresist patterns are arranged on electron beam lithography patterns.

According to FIG. 4A, patterns formed by electron beam lithography may be formed in a narrow region, and may connect patterns formed by photolithography to each other.

According to FIG. 4B, it can be confirmed that there is a portion where pattern end portions of the electron beam lithography and the photoresist patterns are overlapped with each other.

The overlapped regions of the two patterns may be about 3 to 5 µm.

That is, when the photolithography patterns are formed after the electron beam lithography patterns are formed, the photo patterns are formed to overlapped with the region where the first region and the second region are adjacent to each other, so that the electron beam patterns and the photo patterns may be naturally connected to each other.

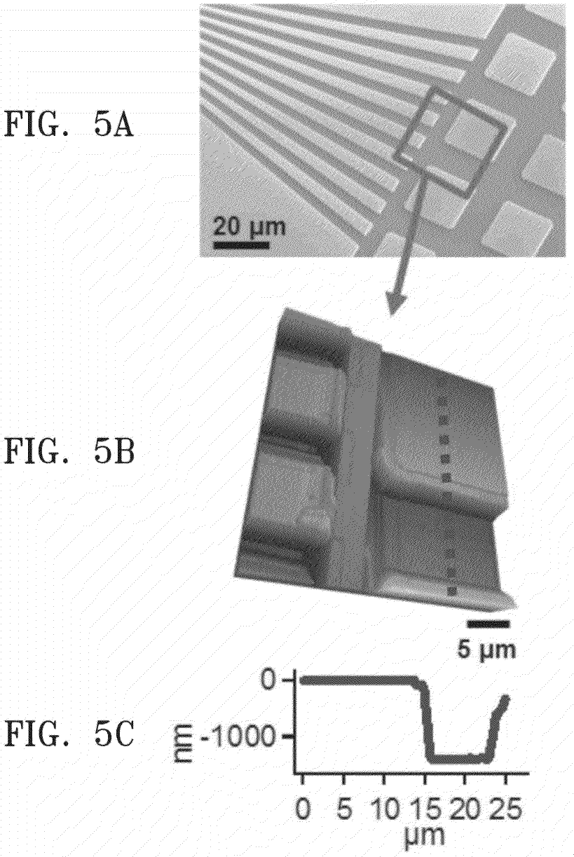

FIGS. 5A to 5C illustrate results of confirming mixed patterns by microscopy.

FIG. 5A is a result of confirming a mixed pattern portion through scanning electron microscopy. FIG. 5B is a result of confirming a mixed pattern portion through atomic force microscopy. FIG. 5C is a result of measuring a depth of a formed channel when a profile of the result confirmed through atomic force microscopy is shown. The depth of the channel is measured to be about 1 µm.

According to the observation result of FIG. 5, it can be confirmed that the electron beam patterns are arranged in the shape of a nanopillar structure, and is formed to be naturally connected to wider photo patterns after the electron beam patterns are terminated.

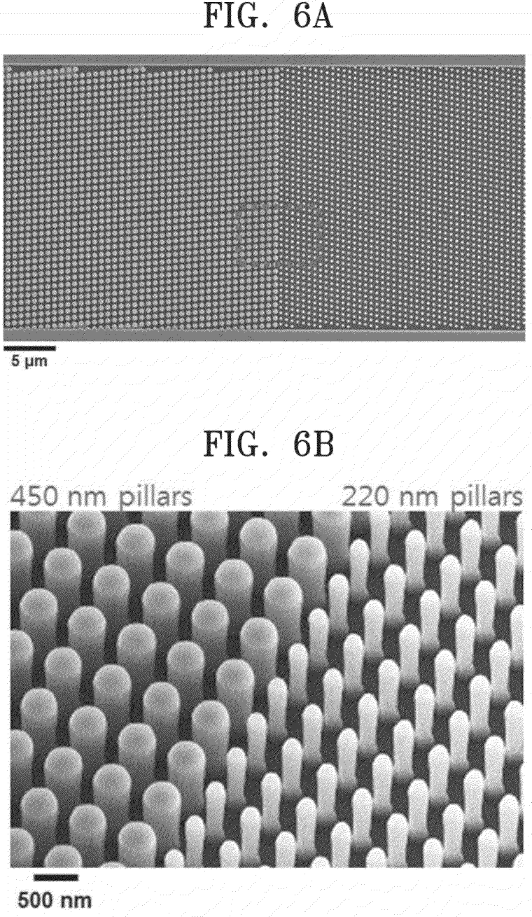

FIGS. 6A to 6B are results confirmed by microscopy by magnifying a middle mixed portion when the process is terminated.

More specifically, FIG. 6A illustrates a result of confirming nanopillar structures through scanning electron microscopy by magnifying the middle portion when the process is terminated. The sizes of the nanopillars may be formed to be similar to the sizes of EVs to be isolated and sorted. According to an example, the sizes of the nanopillars may be designed to be 50 nm to several hundred nanometers. Referring to the case of FIG. 6A, a nanopillar structure of about 400 nm was used for EV isolation, and a nanopillar structure of about 200 nm was used for EV sorting. The nanopillar structure for EV isolation is located at a left side, and the nanopillar structure for EV sorting is located at a right side. FIG. 6B illustrates a result photographed by further magnifying FIG. 6A through scanning electron microscopy and inclining a sample.

According to the manufacturing method of the medical diagnostic chip manufactured according to the exemplary embodiment of FIG. 2, it can be confirmed that the medical diagnostic chip including nanopillar structures having various sizes may be manufactured.

When the nanopillars formed in the manufactured medical diagnostic chip are large, soluble proteins and EVs may be isolated, and when the nanopillars are small, EVs may be sorted by size.

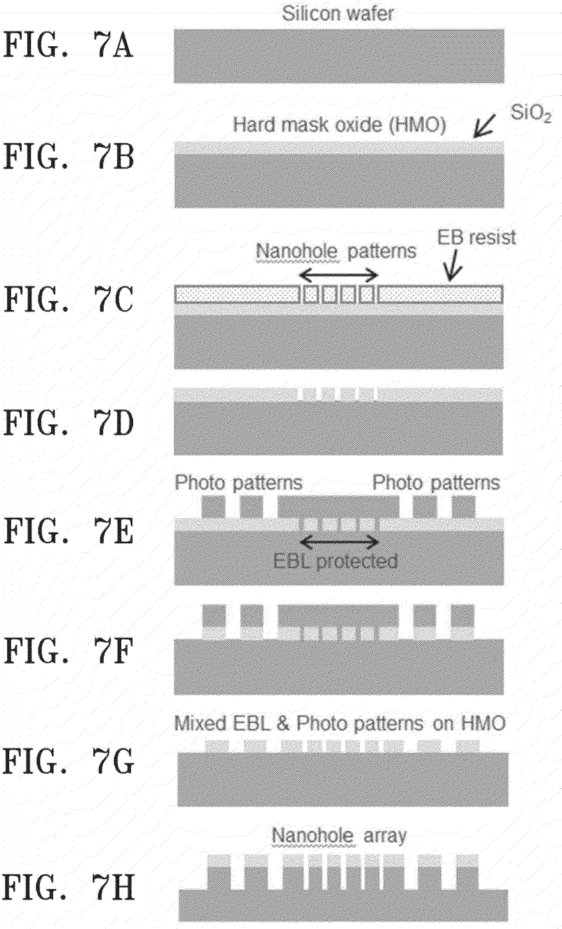

FIGS. 7A to 7H are diagrams sequentially illustrating a method for manufacturing a medical diagnostic chip according to another exemplary embodiment of the present invention.

FIGS. 7A and 7B are the same as the above-described FIGS. 2A and 2B, and thus will be omitted.

Referring to FIG. 7C, first lithography may be performed on a first region by using an electron beam resist. A difference in FIG. 7C from the exemplary embodiment of FIG. 2C is that electron beam patterns may be formed in the form of nanoholes rather than in the form of nanopillars.

In this case, the electron beam resist may be a positive resist. According to FIG. 7C, in contrast to the exemplary embodiment of FIG. 2, nanoholes rather than nanopillars may be formed as the electron beam patterns.

The criteria for sorting the electron beam patterns into nanopillars and nanoholes are as follows.

In the case of forming the electron beam patterns as nanopillar patterns, pillars having nano diameters are formed in the first region by etching the oxide film using the nanopillar type electron beam pattern as an etching mask.

In the case of forming the electron beam patterns as nanohole patterns, nanoholes having nano diameters are formed in the second region by etching the oxide film using the nanohole type electron beam pattern as an etching mask.

FIG. 7D illustrates performing the oxide film etching according to the formed electron beam patterns. After electron beam lithography is performed according to the process of FIGS. 7A to 7D, photolithography is performed.

The photolithography patterns in FIG. 7E are different from the photolithography patterns in FIG. 2E in that photo patterns formed in the first region may be formed to cover(protect) the first region, so that the photolithography process may be performed so as not to affect the first region.

In the exemplary embodiment of FIG. 7, it is necessary to protect the nanohole portion, so that the photo patterns may be formed to protect the already formed electron beam patterns.

In the exemplary embodiment of FIG. 2, since the nanopillars are formed by the nanopillar type electron beam patterns, the formed nanopillar structure may be used for isolating and sorting the device from the medical diagnostic chip. When the photo patterns are formed according to the exemplary embodiment of FIG. 2, there is a need for a process for connecting adjacent portion of the first region and the second region.

On the other hand, in the exemplary embodiment of the process according to FIG. 7, since the nanoholes are formed using the electron beam patterns, the formed chip cannot be used as a medical diagnostic chip itself, but needs to be used as a mold to form the medical diagnostic chip.

Referring to FIG. 7E, it can be seen that the photo patterns are formed by a portion wider than the first region to protect the formed electron beam patterns.

According to FIG. 7F, the oxide film etching is performed according to the formed photo patterns, and according to FIG. 7G, the strip removal of the photoresist is performed to form a nanohole structure. In the exemplary embodiment of FIG. 7, the photoresist may be formed using a positive or negative resist.

According to FIG. 7H, the depth of the formed nanohole structure may be adjusted by additionally performing etching of the substrate. This process may be an optional process.

As described above, the sorting of the device is possible only by using the shape of nanopillars. The medical diagnostic chip formed according to the process embodiment of FIG. 7 is difficult to be used for medical diagnosis only by itself, and the medical diagnostic chip may be formed by using the corresponding chip as a mold.

More specifically, a polymer material may be poured into the nanohole structure completed according to the process of FIG. 7 to form the medical diagnostic chip. The polymer material may be polydimethlysiloxane (PDMS). When a certain time elapses after pouring the polymer material, the polymer material may be hardened. According to an exemplary embodiment, heat may also be applied to harden the polymer material. When the hardened polymer material is separated from the mold, the polymer material may be provided in the form of a chip in which the nanopillars are formed as illustrated in FIG. 2. According to such an exemplary embodiment, there is an advantage in that it is easy to manufacture the medical diagnostic chip by forming the mold at a lower price.

As another method, more specifically, the nanohole structure completed according to the process of the exemplary embodiment of FIG. 7 is converted into the nanopillar structure by a sacrificial process to manufacture a medical diagnostic chip. The sacrificial process refers to a process of covering a material that cannot be etched on the nanohole structure and selectively removing the nanohole structure through an etching gas or an etching liquid. By using the method, it is possible to convert the nanohole structure into a nanopillar structure-based channel structure.

According to an example, it is assumed that a nanohole structure is a silicon (Si) material, a covering material is silicon oxide, and XeF₂ is used as an etching gas. At this time, since XeF₂ as the etching gas has a property of selectively etching only the silicon material, XeF₂ as the etching gas may be used in the sacrificial process. In this case, the silicon material etched by the XeF₂ etching gas corresponds to the sacrificial material.

The example is merely an exemplary embodiment, and various etching gases or etching liquids, substrates, and the like that may be used in the sacrificial process may be provided.

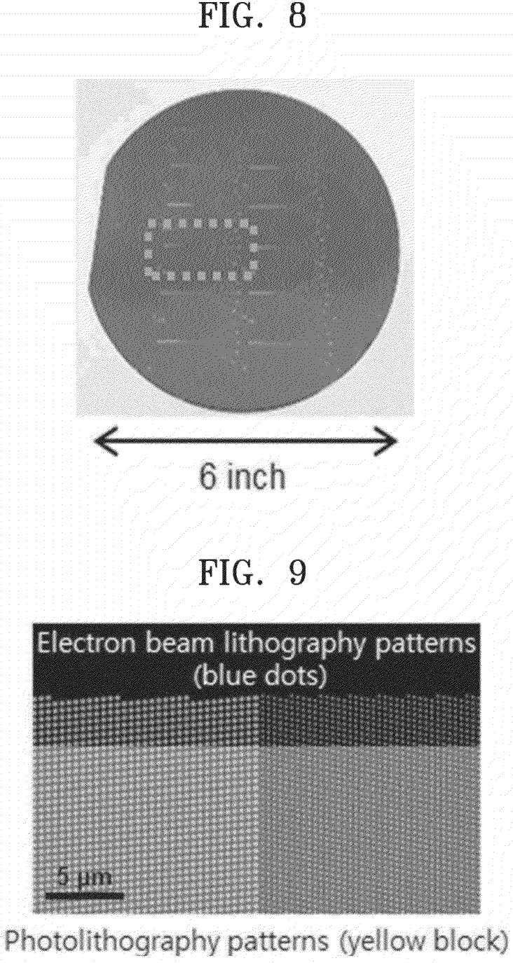

FIG. 8 is a diagram illustrating a medical diagnostic chip manufactured according to the exemplary embodiment in FIG. 7. Referring to FIG. 8, a substrate is formed in a shape having nano holes on the substrate.

FIG. 9 is a diagram for describing a process of FIG. 7E in more detail.

According to FIG. 9, blue dots indicate an example of patterns formed by electron beam lithography, and yellow dots indicate an example of patterns formed by photolithography.

Referring to FIG. 9, by protecting the blue dots, which are patterns formed by electron beam lithography, with the yellow dots, which are patterns formed by photolithography, there is an effect of protecting nanoholes formed by electron beam lithography. In the case of the blue dots that are not protected by the patterns formed by photolithography in FIG. 9, this corresponds to extra nanohole patterns to in preparation for misalign of the yellow photoresist region. As a result, only the blue nanoholes protected from the yellow photoresist are finally left.

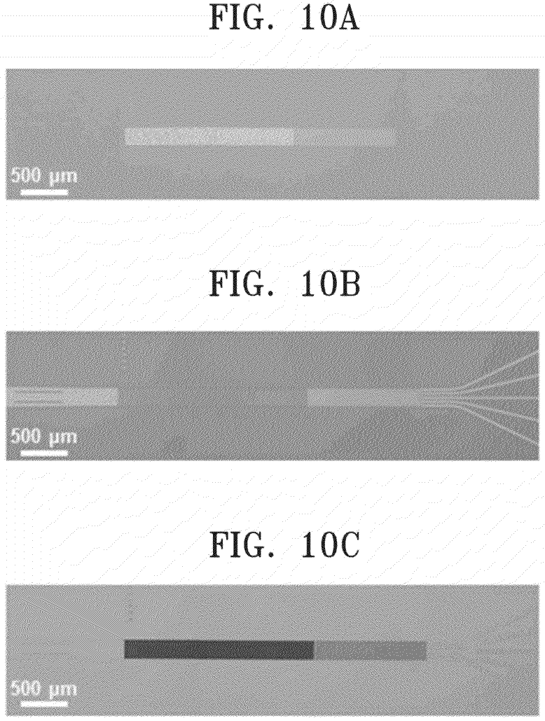

FIGS. 10A to 10C illustrate results in which electron beam lithography patterns and photoresist patterns are arranged.

FIG. 10 is a diagram illustrating observation of electron beam lithography patterns and photolithography patterns by microscopy.

FIG. 10A illustrates a result in which a nanohole structure is implemented by electron beam lithography, FIG. 10B illustrates a result in which the nanohole structure is covered and protected by a photoresist, and FIG. 10C illustrates a result in which the nanohole structure implemented by electron beam lithography and a microfluidic structure implemented by photolithography are mixed.

That is, according to FIG. 10, it can be confirmed that the hole structure may be easily formed by protecting the nanohole structure by the photoresist patterns.

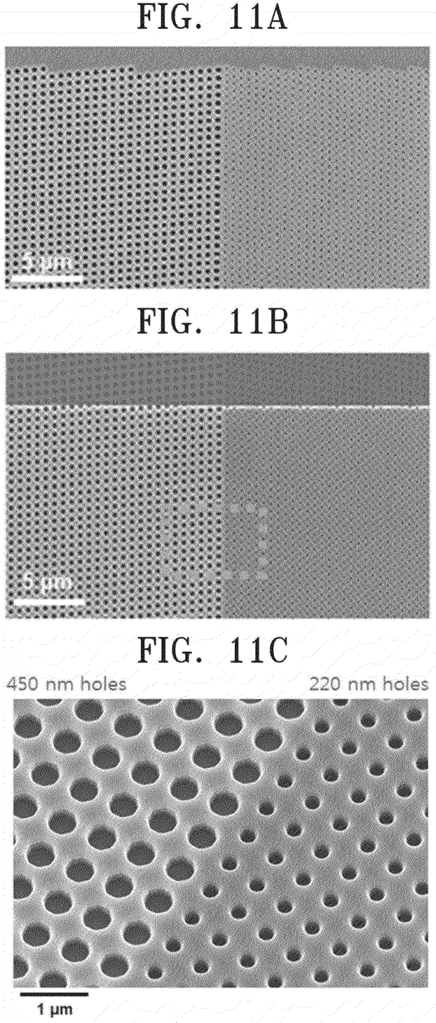

FIGS. 11A to 11C illustrate results of confirming mixed patterns by microscopy.

FIGS. 11A and 11B are results of observing a nanohole structure implemented by electron beam lithography through scanning electron microscopy, and FIG. 11C is a result of magnifying and observing a structure in which a nanohole structure and a microfluidic structure are mixed.

Referring to FIG. 11, it can be seen that nanoholes having various sizes are formed.

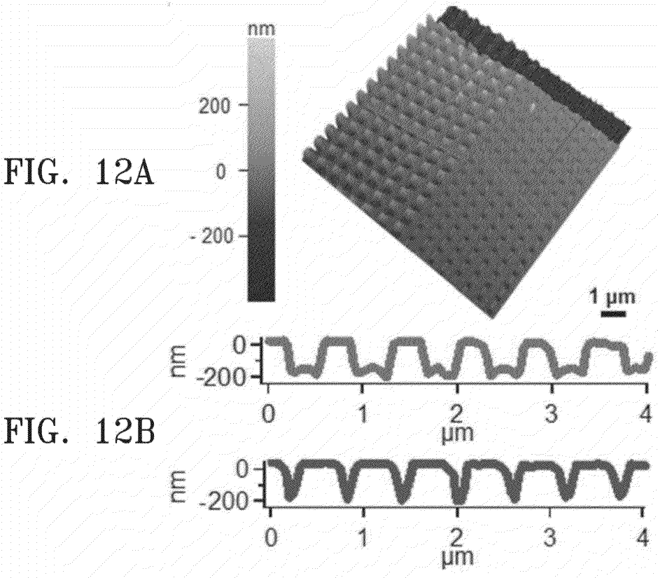

FIGS. 12A and 12B illustrate a result of confirming a nanohole structure by microscopy and a result of observing the depths of nanoholes.

FIG. 12A is a result of observing a nanohole structure through atomic probe microscopy, and FIG. 12B illustrates a result of observing depths of the nanohole structure through surface profiling.

According to FIG. 12B, it can be confirmed that the depth of the nanoholes is observed at a depth of about 200 nm.

Referring to FIGS. 7 to 12, it can be confirmed that the substrate having the nanohole structure may be formed using such a process, and it can be seen that when a medical diagnostic chip is formed by using the substrate as a mold, nanopillars having heights corresponding to the depths of the nanoholes will be formed.

It is possible to mix and form patterns having nanopillars and micropillars by using the methods for manufacturing the medical diagnostic chip as described above. According to the size of an endoplasmic reticulum to be isolated, an interval and the shape of the electron beam lithography patterns may be adjusted, and the intervals of the photolithography patterns may also be adjusted.

In addition, in the example, an example of forming only a nanopillar structure on one substrate or forming only a nanohole structure on one substrate was illustrated, but it is also possible to simultaneously form the nanopillar structure or the nanohole structure on one substrate.

According to an exemplary embodiment of the present invention, the nanopillar structure may be directly manufactured on a silicon wafer and used as a medical diagnostic chip by itself. According to the method manufactured according to another exemplary embodiment of the present invention, it is possible to form a chip having a polymer nanopillar structure by using a silicon wafer formed to have a nanohole structure as a template or a mold. In addition, a chip may be formed through a process of converting a nanohole structure into a nanopillar structure by a sacrificial process. According to the present invention, it is possible to implement nanometer-level small structures on a large area of millimeters or more.

According to the present invention, pillars having micrometer-level sizes may be used as a means for flowing samples, and the samples may be isolated or sorted through the nanopillar structure. The isolating or sorting of the samples may vary depending on the size of the nanopillar structure.

According to an example, the samples may be isolated through the nanopillars having a size of 400 nm, and the samples may be sorted by size through the nanopillars having a size of 200 nm. This may be processed by patterns formed by the first lithography method.

It is to be understood that the exemplary embodiments are presented to assist in understanding of the present invention, and the scope of the present invention is not limited, and various modified exemplary embodiments thereof are included in the scope of the present invention. The technical protection scope of the present invention should be determined by the technical idea of the appended claims, and it should be understood that the technical protective scope of the present invention is not limited to the literary disclosure itself in the appended claims, but the technical value is substantially affected on the equivalent scope of the invention.

1. A method for manufacturing a medical diagnostic chip using a mixed lithography method, comprising:
forming first patterns in a first region using a first lithography; and
forming second patterns in a second region using a second lithography,
wherein a part of the second patterns is formed in a part of the first region among the region where the first region and the second region are adjacent to each other.

2. The method for manufacturing the medical diagnostic chip of claim 1, wherein
the first lithography is electron beam lithography, and
the second lithography is photolithography.

3. The method for manufacturing the medical diagnostic chip of claim 2, wherein the forming first patterns in the first region using the electron beam lithography comprises forming nanopillar pattern.

4. The method for manufacturing the medical diagnostic chip of claim 3, wherein the forming first patterns in the first region using the electron beam lithography comprises forming the patterns using a positive electron beam resist.

5. The method for manufacturing the medical diagnostic chip of claim 3, wherein the forming a part of second patterns in the portion of the first region among the region where the first region and the second region are adjacent to each other comprises forming the second patterns using the second lithography as to overlap with an edge portion of the first region among the region where the first region and the second region are adjacent to each other.

6. The method for manufacturing the medical diagnostic chip of claim 5, wherein the forming second patterns in the second region using the second lithography comprises forming the patterns using a positive or negative photoresist.

7. A method for manufacturing a medical diagnostic chip using a mixed lithography method, comprising:
forming first patterns in a first region using first lithography; and
forming second patterns in a second region using second lithography,
wherein the forming second patterns in the second region using the second lithography includes forming the second patterns so that the first patterns are protected.

8. The method for manufacturing the medical diagnostic chip of claim 7, wherein
the first lithography is electron beam lithography, and
the second lithography is photolithography.

9. The method for manufacturing the medical diagnostic chip of claim 8, wherein the forming first patterns in the first region using the electron beam lithography comprises forming nanoholes.

10. The method for manufacturing the medical diagnostic chip of claim 9, wherein the forming first patterns in the first region using the electron beam lithography comprises forming the first patterns using a positive electron beam resist.

11. The method for manufacturing the medical diagnostic chip of claim 9, wherein the forming second patterns so that the first patterns are protected comprises forming the second patterns so as not to affect the first patterns formed in the first region.

12. The method for manufacturing the medical diagnostic chip of claim 11, wherein the forming second patterns in the second region using the second lithography comprises forming the second patterns using a positive or negative photoresist.

13. The method for manufacturing the medical diagnostic chip of claim 12, further comprising:
pouring a polymer material into the formed chip;
waiting for a predetermined time until the polymer material is hardened; and
inverting the hardened polymeric material.

14. A medical diagnostic chip manufactured by the manufacturing method of claim 1.

15. A medical diagnostic chip manufactured by the manufacturing method of claim 7.
